(19) United States
(12) Patent Application Publication
(10) Pub. No.: US 2016/0213692 A1
(43) Pub. Date: Jul. 28, 2016

(54) ADVANCED METHOD OF TREATMENT AND MANAGEMENT OF CHRONIC HEPATITIS C INFECTION

(71) Applicant: Umm Al-Qura University, MAKKAH (SA)

(72) Inventors: ADEL GALAL AHMED EL-SHEMI, Makkah (SA); Bassem Amr Refaat, JEDDAH (SA); Ahmed Mohammed Ashshi, MAKKAH (SA)

(21) Appl. No.: 14/605,580

(22) Filed: Jan. 26, 2015

(51) Int. Cl. A61K 31/593; A61K 38/21; A61K 31/7056

(52) U.S. Cl. CPC A61K 31/593; A61K 31/7056; A61K 38/212

(57) ABSTRACT

Chronic hepatitis C (CHC) infection is associated with the development of liver fibrosis, cirrhosis and hepatocellular carcinoma. Coherently, the current study is designed to determine the therapeutic value of vitamin D as a supplement to the currently recommended treatment in Saudi Arabia patients diagnosed with CHC and the possible correlation between vitamin D status and the target molecules in the prediction of viral response to current therapy for CHC. Additionally the study also explores a role of vitamin D supplementation in reducing side effect of currently used therapy for CHC infection.

### FIELD OF TECHNOLOGY

This disclosure generally relates to the treatment and management of viral infection. More specifically, the disclosure relates to a supplementation therapy treatment and early identification of non-responders to manage patients with viral infection such as chronic hepatitis C infection.

### BACKGROUND

Hepatitis C virus (HCV) is a major human pathogen infecting millions of people world-wide. Chronic hepatitis C (CHC) infection is associated with the development of liver fibrosis, cirrhosis and hepato cellular carcinoma. CHC infection shows intermediate endemicity in Saudi Arabia with an estimated seroprevalence of 5.9% and the predominant viral genotypes are 4 and 1, which are difficult to cure and require longer duration of treatment. Currently, the prediction of treatment outcome and the identification of non-responders are based on viral genotype and kinetics. However, the actual predictive value of these factors is low and response to therapy is highly unpredictable. A number of host factors have been recently suggested to potentially influence the immune response, liver biology and the treatment outcome in CHC infection.

The patient suffering from CHC infection have currently no effective therapeutic alternative and thus are at a significant risk of developing complication of advanced liver disease as well as markedly increased risk of hepatocellular carcinoma. Further, the treatment outcome still needs to be explored further in patients from Saudi Arabia.

### SUMMARY

The present invention discloses a supplementation therapy for a treatment of a patient with a CHC infection. Further, the present invention discloses a possible correlation between the supplementation therapies as disclosed with a routine therapy given to a patient diagnosed with a CHC infection. The present disclosure also relates to a role of supplemental therapy in prevention of a side effect of a routine therapy.

In one embodiment, a patient will be diagnosed with a CHC infection. Diagnosis of the CHC infection may be based on the diagnosis of one or more signs and/or symptoms such as an elevated alanine aminotransferase (ALT), a positive test for an anti-HCV antibody, a presence of CHC infection as demonstrated by a positive test for a CHC-RNA, a clinical signal of a chronic liver disease, a hepacellular damage among other routinely used diagnostic parameters. In another embodiment, a patient will be diagnosed with a CHC infection as per their family history, past ailments and diagnosis along with other related parameters. In most embodiments, a patient from Saudi Arabia (SA) will be diagnosed with a CHC infection by analyzing a sign and/or a symptom as disclosed above.

In one embodiment, plurality of patients diagnosed with the CHC infection will be segregated into ‘n’ number of groups preferably 4 groups such as group 1, group 2, group 3 and group 4. As disclosed group 1 comprise of plurality of patients diagnosed with a CHC infection and receiving no therapy. Group 2 comprise of plurality of patients diagnosed with a CHC infection and receiving a routine therapy. The routine therapy may comprise of a weekly injection of a pegylated interferon-α (Peg-INFα) and/or twice daily weight based dose of a ribavirin (RBV). Group 3 comprise of plurality of patients diagnosed with a CHC infection and receiving the routine therapy along with a supplemental therapy and group 4 comprise of plurality of patients diagnosed with a CHC infection and receiving the supplemental therapy.

In one embodiment, the weekly injection can be of Peg-INFα 2a whereas in another embodiment weekly injection can be of Peg-INFα 2b. Also the routine therapy as disclosed may comprise of weekly injection of Peg-INFα alone or in combination with RBV.

In one embodiment, the supplemental therapy as disclosed may comprise of administration of a vitamin D such as cholecalciferol (4000 IU/ml to 5000 IU/ml) with a final concentration of 700 IU/ml to 1500 IU/ml. In another embodiment, the supplemental therapy as disclosed may also comprise of administration of a vitamin D 3 along with a thymoquinone (TQ).

In another embodiment, plurality of patients diagnosed with CHC infection may be segregated into 8 groups wherein group 1 comprise of plurality of patients diagnosed with a CHC infection and receiving no therapy (Control group); group 2 comprise of plurality of patients diagnosed with a CHC infection and receiving the Peg-INFα (P group); group 3 comprise of plurality of patients diagnosed with a CHC infection and receiving Peg-INFα along with vitamin D (PD group); group 4 comprise of plurality of patients diagnosed with a CHC infection and receiving Peg-INFα along with RBV (PR group); group 5 comprise of plurality of patients diagnosed with a CHC infection and receiving Peg-INFα and RBV along with vitamin D (PRD group); group 6 comprise of plurality of patients diagnosed with a CHC infection and receiving the RBV (R group); group 7 comprise of plurality of patients diagnosed with a CHC infection and receiving the RBV along with vitamin D (RD group); group 8 comprise of plurality of patients diagnosed with a CHC infection and receiving vitamin D only (Vit D group). The study may be carried out for a period anywhere in between 2 weeks and 10 weeks. In most embodiments, the study may be carried out for a period of 5 weeks. At the end of the study period different biological and physiological parameters will be determined.

In one embodiment, following completion of study plan, a blood sample may be collected from the patient followed by centrifugation to get a serum sample. In another embodiment, the serum sample will be analyzed for a vitamin D and an erythropoietin (EPO) concentration. The blood sample may also be collected before the start of the study plan and in between the study plan to analyze the vitamin D and EPO concentrations.

In one embodiment, a kidney specimen may also be collected from the said patients before the start of the study plan, in between the study plan and at the end of the study plan. In another embodiment, a total protein level and a hematological profile may be evaluated in the kidney specimen. Further, study will also be done to evaluate a renal and a liver function. Hematological profile as disclosed may comprise of evaluation of a hemoglobin concentration (Hb conc.), a RBC's count, a packed cell volume (PCV), a mean corpuscular volume (MCV), a mean corpuscular hemoglobin (MCH) and a mean corpuscular hemoglobin concentration (MCHC), a hematocrit (HCT), a red cell distribution width (RDWT), neutophil, lymphocyte, monocyte, eosinophil, basophil, a platelets count, a mean platelet volume (MPV) and an erythrocyte sedimentation rate (ESR).

In one embodiment, a study may also be done to evaluate therapy induced side effect. In another embodiment, a study will be done to evaluate routine therapy induced side effect and the role of supplemental therapy to reduce or prevent them. The side effects may include evaluating for signs of anemia.

In one embodiments, plurality of patients diagnosed with the CHC infection will be segregated into ‘n’ number of groups preferably 4 groups such as group I, group II, group III and group IV. Group I as disclosed may comprise of plurality of patients diagnosed with a CHC infection and receiving a routine therapy for the CHC infection along with a supplementation therapy comprising of vitamin D therapy (such as vitamin D3). Patients will receive vitamin D3 supplementation at a dose level ranging from 500 IU/day to 3000 IU/day with a target serum level>20 ng/ml preferably with a target serum level>32 ng/ml. Group II may comprise of plurality of patients diagnosed with the CHC infection and will receive the routinely used therapy for the CHC infection along with a thymoquinone (TQ) supplementation therapy. The TQ supplementation therapy as disclosed comprise of administering the TQ at a dose range of 5 mg/Kg/day to 40 mg/Kg/day. Group III may comprise of a plurality of patients diagnosed with the CHC infection and receiving the routinely used therapy for the CHC infection along with the supplementation therapy comprising vitamin D (preferably vitamin D3) and TQ. Patients will receive vitamin D3 supplementation at a dose level ranging from 500 IU/day to 3000 IU/day with a target serum level>20 ng/ml preferably with a target serum level>32 ng/ml. TQ supplementation therapy as disclosed comprise of administering TQ at a dose range of 5 mg/Kg/day to 40 mg/Kg/day. Further, group IV as disclosed may comprise of patients diagnosed with the CHC infection and receiving routinely used therapy for the CHC infection only. No supplementation therapy is provided to patients in group IV. Group IV as disclosed may be regarded as a control group for the study group.

In one embodiment, vitamin D3 and TQ in the supplementation therapy may be administered intravenously, intradermaly, subcutaneously, epicutaneously, epidermally, intramuscularly, intraperitoneally or other preferred mode of administration. In another embodiment, vitamin D3 and TQ in supplemental therapy may be administered in the form of a pill, a capsule, a tablet, a liquid or other preferred pharmaceutical formulation.

In one embodiment, a further study may include evaluating a serological marker before the initiation of the supplementation therapy schedule, in between the therapy schedule and following the completion of study schedule in the patient diagnosed with CHC infection. In another embodiment, a further study may include determine a blood concentration of vitamin such as a vitamin D3 before the initiation of the supplementation therapy schedule, in between the therapy schedule and following the completion of study schedule in the patient diagnosed with the CHC infection. In another embodiment, a further study may include a genotypic study performed before the initiation of the therapy schedule, in between the therapy schedule and following the completion of study schedule in the patients diagnosed with the CHC infection. A serological marker as disclosed may comprise of evaluation of an activin and their binding protein, interferon gamma-inducible protein (IP-10), interleukin (IL-10) among other serological marker. Genotypic study as disclosed comprise of studies on genetic variation (if any) in host vitamin D receptor (VDR) gene and interleukin-28B (IL-28 B) gene among other related genes. In most embodiments, a serological marker analysis, a vitamin status evaluation and a genotypic study will be done in patients (especially in Saudi Arabia) diagnosed with the CHC infection before the initiation of the therapy schedule.

Also, disclosed in the present invention is a development of a highly sensitive and a specific marker to analyze the supplementation treatment outcome to allow identification of a non-responder which may help in planning a treatment protocol and duration of treatment protocol for a patient diagnose with a CHC infection.

In one embodiment, administering supplementation therapy comprises: diagnosing patients with a CHC infection; administering a regularly used anti-CHC infection drug; administering a supplementation therapy wherein the supplementation therapy comprise of a vitamin D dose and analyzing a CHC infection marker. In another embodiment, administering supplementation therapy comprises: diagnosing patients with a CHC infection; administering a regularly used anti-CHC infection drug; administering a supplementation therapy wherein the supplementation therapy comprise of a combination of a vitamin D dose and a TQ and analyzing a CHC infection marker.

In one embodiment, a therapeutic value of a vitamin (such as vitamin D3) supplementation will be evaluated in improving the treatment outcome in patients diagnosed with CHC infection. In another embodiment, a therapeutic value of a TQ supplementation will be evaluated in improving the treatment outcome in patients diagnosed with CHC infection.

In one embodiment, an estimation of a hematological profile, liver function, renal function, a serological marker, a vitamin status and a genotype study for a gene polymorphism before the initiation and following completion of supplementation therapy schedule will lead to an observation of a non-responder to the supplementation therapy. In another embodiment, an estimation of a hematological profile, liver function, renal function, serological marker, a vitamin status and a genotype study for gene polymorphism before the initiation and following completion of supplementation therapy schedule will lead to a precise optimization of the supplementation therapy protocol and thereby duration of supplementation and routine therapy combination to a patient with a CHC infection. The present invention will help in development of highly sensitive and specific marker (s) for treatment outcome, evaluating a side effect and thus may allow early and accurate identification of non-responders and precise optimization of treatment protocol and duration of supplementation therapy in CHC infection.

Other features will be apparent from the accompanying figures and from the detailed description that follows.

### BRIEF DESCRIPTION OF THE DRAWINGS

Example embodiments are illustrated by way of example and no limitation in the tables and in the accompanying figures, like references indicate similar elements and in which:

FIG. 1(a) shows erythrocyte count, FIG. 1(b) shows hemoglobin concentration, FIG. 1(c) shows kidney erythropoietin; and FIG. 1(d) shows serum erythropoietin in the different study groups.

FIGS. 2(a), 2(b) and 2(c) shows results of correlation study in the different study groups.

### DETAILED DESCRIPTION

The present invention study the role of a supplementation therapy with the currently (regularly) used therapy for the treatment of patients diagnosed with a CHC infection. The supplementation therapy as disclosed comprise of administration of a vitamin (such as a vitamin D and/or vitamin D3) and/or a TQ alongside the regular therapy for CHC infection especially in patients from Saudi Arabia (SA). Infection with hepatitis C virus is a global health problem associated with the development of liver fibrosis, cirrhosis and hepatocellular carcinoma. There are at least 180 million infected persons worldwide with 3 to 4 million newly acquired infections each year.

CHC infection is currently treated with a weekly injection of PEG-IFN-α-2a or -2b plus a twice-daily weight-based dose of RBV. However, this standard of care is considered suboptimal because of its long duration, inability to cure about half of all patients, expenses, and numerous potentially severe side effects, which lead to dose reduction and/or premature termination of treatment. The major adverse effects associated with the currently used medical treatment include hematological problems (anemia, thrombocytopenia) and endocrinological side effects, particularly affecting the thyroid gland.

Almost all patients treated with peg-INF-α and RBV experience one or more adverse events during the course of therapy. Adverse events are a major reason that patients decline or stop therapy altogether. In the registration trials of peg-INF-α-2a and 2b plus RBV, 10% to 14% of patients had to discontinue therapy due to an adverse event (Averhoff et al., 2012; Ghany et al, 2009). Hematological abnormalities are major side effects of Peg-IFN-α based therapy and the most common and important one is the development of anemia (Sandokji et al., 2003; Garcia et al., 2012; McHutchison et al, 2007; Kowdley 2005; Keeffe and Kowdley, 2005).

Several mechanisms for the development of anemia during Peg-INF-α based therapy have been suggested. Peg-INF-α may lead to anemia by suppressing the proliferation of progenitor cell, increasing erythroid precursor cells destruction, autoimmune hemolytic reactions, and reducing renal function (Kurschel et al, 1991; Sacchi et al, 1995; Tarumi et al, 1995; Kato et al, 2003). Alternatively, RBV induces hemolytic anemia in a dose-dependent manner, which is believed to be exacerbated by Peg-INF-α (Sulkowski et al, 2004; Tanaka et al, 2005; Morello et al, 2008). RBV may also lead to anemia by down-regulating the expression of erythropoietin receptors (Kowdley, 2005; Van Vlierbergh, 2001; De Franceschi, 2000) and both drugs were also associated with a decrease in serum erythropoietin hormone (EPO) (Balan et al, 2005; Martin and Jensen, 2008). Hence, Peg-INF-α based therapy could induce anemia by hemolysis and/or suppression of erythropoiesis.

The initial clinical management of anemia associated with Peg-INF-α based therapy was the reduction of RBV dosage. However, several reports have demonstrated a correlation between response rate and higher RBV dose (McHutchison et al, 2002; Shiffman et al, 2007a, 2007b; Poordad et al, 2013). Therefore, the use of erythropoiesis-stimulating agents (ESA), such as EPO, and blood transfusion have been introduced as alternatives for RBV dose reduction to support anemic patients during the course of treatment (Poordad et al, 2013; Sulkowski et al, 2013).

Currently, the prediction of treatment outcome and the identification of non-responders in patients infected with chronic hepatitis C (CHC) are based on viral genotype and kinetics. However, the actual predictive value of all these factors is still low and response to therapy remains highly unpredictable. Recently, a number of host factors have been suggested to potentially influence the immune response, liver biology and the treatment outcome in CHC infection. In this regard, the serum level of vitamin D, expression levels of activins and their binding proteins, interferon gamma-inducible protein-10 (IP-10) and inteleukin-10 (IL-10), as well as genetic variation in host interleukin-28B gene (IL-28B gene polymorphism) have been reported to regulate the host immune response and/or treatment outcome in CHC infection.

Vitamin D has been shown to play important roles in the regulation of several systems beside its role in bone and mineral metabolism (Deicher and Horl, 2005; Sim et al, 2010; Rianthavorn and Boonyapapong 2013). Vitamin D regulates the process of erythropoiesis by stimulating erythroid progenitor cells in a synergistic fashion with other hormones and cytokines, including EPO, and it has been reported that vitamin D is crucial for normal red blood cell production (Alon et al, 2002). The prevalence of anemia and the use of ESA have been found to be negatively correlated with serum Vitamin D levels regardless of kidney function in the general population (Sim et al, 2010). The role of vitamin D in erythropoiesis has also been suggested by several clinical observations, especially in hemodialysis patients, where administration of Vitamin D has been has been associated with dose reductions in ESA and increased reticulocytosis (Albitar et al, 1997; Saab et al, 2007). Furthermore, vitamin D3 (calcitriol), in synergism with EPO, increases the production of EPO receptor at the mRNA and protein levels in vitro (Alon et al, 2002).

Little is known about the role of Vitamin D as a prophylactic/treatment for anemia associated with Peg-INF-α based therapy. We therefore hypothesis that vitamin D3 supplementation may provide protection against anemia associated with the current treatment of CHC. The present work is a preclinical study to measure the effect of vitamin D3 (cholecalciferol) supplementation with Peg-INF-α and RBV on erythrocyte indices and concentrations of EPO in serum and kidney of normal rat.

Further, the study plan may also comprise of combine supplemental therapy comprising supplementation with vitamin D along with Thymoquinone (TQ), the main active biological compound of Nigella Sativa herbal plan with promising immunomodulatory and antiviral activity, against CHC infection.

To carry out the disclosed invention, patients especially from SA will be first diagnosed with CHC infection. Diagnosis will be carried out by routine tests done in the laboratories world-wide. The diagnosis criteria may include symptoms including (but not limited to) an elevated alanine aminotransferase (ALT), a positive test for an anti-HCV antibody, a presence of CHC infection as demonstrated by a positive test for a CHC-RNA, a clinical signal of a chronic liver disease, a hepacellular damage among other routinely used diagnostic parameters.

Following the diagnosis of CHC infection, patients will be segregated into ‘n’ number of groups. In the present disclosure, patients will be divided into 4 groups namely group 1, 2, 3 and 4. Group 1 comprise of plurality of patients diagnosed with a CHC infection and receiving no therapy. Group 2 comprise of plurality of patients diagnosed with a CHC infection and receiving a routine therapy. The routine therapy may comprise of a weekly injection of a pegylated interferon-α (Peg-INFα) and/or twice daily weight based dose of a ribavirin (RBV). Group 3 comprise of plurality of patients diagnosed with a CHC infection and receiving the routine therapy along with a supplemental therapy and group 4 comprise of plurality of patients diagnosed with a CHC infection and receiving the supplemental therapy.

Further patients may also be divided into 4 groups namely group I, group II, group III and group IV. Group I comprise patients diagnosed with CHC infection and receiving the routinely used therapy to treat CHC infection along with a supplementation therapy comprising of vitamin D therapy (preferably vitamin D3). Patients will receive vitamin D3 supplementation at a dose level ranging from 500 IU/day to 3000 IU/day with a target serum level>20 ng/ml preferably >32 ng/ml. Further, group II comprise of patients diagnosed with CHC infection and will receive the routinely used therapy to treat CHC infection along with thymoquinone (TQ) supplementation therapy. TQ supplementation therapy as disclosed comprise of administering TQ at a dose range of 5 mg/Kg/day to 40 mg/Kg/day. Group III comprise of patients diagnosed with CHC infection and receiving the routinely used therapy to treat CHC infection patients along with supplementation therapy comprising vitamin D (preferably vitamin D3) and TQ. Patients will receive vitamin D3 supplementation at a dose level ranging from 500 IU/day to 3000 IU/day with a target serum level>20 ng/ml. TQ supplementation therapy as disclosed comprise of administering TQ at a dose range of 5 mg/Kg/day to 40 mg/Kg/day. Group IV comprise of patients diagnosed with CHC infection and receiving routinely used therapy to treat CHC infection only. No supplementation therapy is provided to patients in group IV. Group IV as disclosed may be regarded as a control group for the present study plan.

### Drugs

Pegylated interferon-α-2a (Pegasys®, Hoffmann-La Roche, Nutley, N.J.) was used. The ready to use syringe contains 180 μg/0.5 ml. Ribavirin capsules (Viracure®, 6 October Pharm, Egypt) were used and each capsule contains 400 mg of ribavirin. Vitamin D3 (cholecalciferol 4500 IU/mL) oral drops (VitD3, Novartis International AG, Basel, Switzerland) was used in the study.

### Study Design

A total of 80 male Wistar rats weighing 250-300 gm were used. All animals received humane care during the study protocol and during sacrifice. The animals were divided equally into 8 groups as follow: The first group included 10 rats and they served as ‘Control group’, the second group consisted of those that only received Peg-INF-α ‘P group’, the third group received Peg-INF-α+VitD ‘PD group’, The fourth group received Peg-INF-α+RBV ‘PR group’, The fifth group received Peg-INF-α+RBV+VitD ‘PRD group’, the sixth group received RBV only ‘R’ group, the seventh group received RBV+VitD ‘RD group’ and the last group consisted of rats that received vitamin D3 only ‘VitD group’.

### Treatment Protocol

The study duration was 5 weeks. Peg-INF-α-2a was prepared by diluting the content of a full syringe (180 μg/0.5 ml) in 9.5 ml sterile normal saline to prepare a final volume of 10 ml and the final concentration was 18 μg/ml. Each rat in the ‘P’, ‘PD’, ‘PR’ and ‘PRD’ groups received a weekly subcutaneous injection of 0.33 ml (6 μg/rat) for a total of 4 injections. The drug was prepared fresh on the day of use. One capsule of RBV (400 mg) was dissolved in 50 ml saline every day of the experiment and each rat in the ‘PR’, ‘PRD’, ‘RBV’ and ‘RD’ received 0.5 ml (4 mg/day) orally for the whole length of the study similar to the highest dose of the drug recommended from human during CHC treatment (12 mg/kg [1200 mg for body weight≧75 Kg]) (Ghany et al, 2009). Cholecalciferol (4500 IU/mL) was prepared by adding 4 ml to 16 ml saline every morning to form a final concentration of 1000 IU/mL. Each rat in the ‘PD’, ‘PRD’, ‘PR’ and ‘VitD’ groups received 0.5 ml/day (500 IU/day) for the full study duration. Cholecalciferol, and its dose, was chosen over calcitriol, the hormonal form of vitamin D, to avoid the risk of soft tissue calcification (Salum et al, 2012). Following 4 injections, the rats were sacrificed in the fifth week at the time of the 5^{th} injection would have been given. RBV and vitamin D3 were continued till the day before sacrifice.

### Types of Sample

All Rats were sacrificed on the same day under anesthesia using diethyl ether (Fisher Scientific UK Ltd, Loughborough, UK) a week after the last injection. 1 ml of blood was collected on EDTA for CBC and 3 ml of blood were collected in plane tube immediately after cutting the vena cava. Blood samples in plane tubes were centrifuged and the serum was stored in −20° C. for routine biochemistry and to measure serum concentrations of 25-OH vitamin D and EPO.

A specimen weighing 1 gm from both kidneys (0.5 gm from each) was obtained from each animal and it was used immediately for protein extraction using 3 ml of RIPA lysis buffer containing protease inhibitors (Santa Cruz Biotech, USA) and electrical homogeniser. All samples were centrifuged at 14000 rpm for 30 minutes and small aliquots (0.5 ml) of the resultant supernatant were placed in eppendorf tubes and stored in −20° C. till processed to measure the levels of candidate proteins in kidney using ELISA.

### Measurement of Extracted Protein Concentrations

The concentrations of the total proteins extracted from the kidney specimens were measured using the BioSpec-nano (Shimadzu Corporation, Japan) at 280 OD. All protein samples were diluted using normal sterile saline to make a final concentration of 500 μg/ml of total protein.

### Determination of Haematological Profile

Whole blood samples (1 ml) collected on EDTA were processed on Sysmex XS 500 (Sysmex, IL, USA) for the measurement of hemoglobin concentrations, RBCs count, packed cell volume (PCV), mean corpuscular volume (MCV), mean corpuscular hemoglobin (MCH) an mean corpuscular hemoglobin concentration (MCHC).

### Liver and Renal Function Parameters

The quantitative measurement of serum liver enzymes (ALP, ALT and AST), total and indirect bilirubin, creatinine and urea was done using Cobas e411 (Roche Diagnostics International Ltd, Switzerland) according to the manufacturer protocol.

### Enzyme Linked Immunosorbant Assay (ELISA)

ELISA was used for quantitative measurement of serum total 25-OH Vitamin D (Dialab, Objekt, Austria) and, serum and kidney EPO concentrations (Cusabio, Hubei, China). All samples were processed in duplicate on a fully automated ELISA system (Human Diagnostics, Germany) and according the manufacturers' instructions. The optical density of the plates was measured within 5 minutes at 450 nm as recommended by the manufacturers.

### Statistical Analysis

Statistical analysis of the results was performed using SPSS version 16. Normality and homogeneity of data were assessed with the Kolmogorov and Smirnoff test and Levene test, respectively. One way ANOVA followed by LSD post hoc test were used to compare between the different groups. Correlations were determined using Pearson's test. P value<0.05 was considered significant.

### Routine Biochemistry

There was no significant difference (P>0.05) using one way ANOVA between the different study groups in body weight, liver enzymes, total bilirubin, indirect bilirubin and renal function parameters (Table 1a and 1b). However, serum concentrations of total 25-OH Vitamin D were significantly higher in the groups that received cholecalciferol compared to the other study groups (Table 1a and 1b).

### Erythrocyte Indices

The administration of Peg-INFα-2a alone (P group) did not affect any of the erythrocyte parameters compare to ‘Control’ group′ (p>0.05). The addition of RBV to Peg-INF-α(PR group), significantly decreased the RBCs count (7.9±0.6×10⁻⁶/μl; p=0.002) and hemoglobin concentration (13.8±0.7 g/dL; p=0.0001) compared to control (Table 2a and 2b). Additionally, the combination of RBV with Peg-INF-α (PR group) significantly decreased the hemoglobin compared to the Peg-INF-α only (P) group. The lowest RBCs count (7.58±0.7×10⁻⁶/0) and hemoglobin concentration (13.1±1 gm/dL) was detected with RBV only (R group) and its was significantly lower compare to the control group (p=0.0004) and ‘P group’ (p=0.003). However, there was no significant difference between the ‘PR’ and ‘R’ groups. Additionally, there was no significant difference between all study groups in PCV, MCV and MCH (Table 2a and 2b), suggesting that the induced anemia by the drugs is normocytic normochromic.

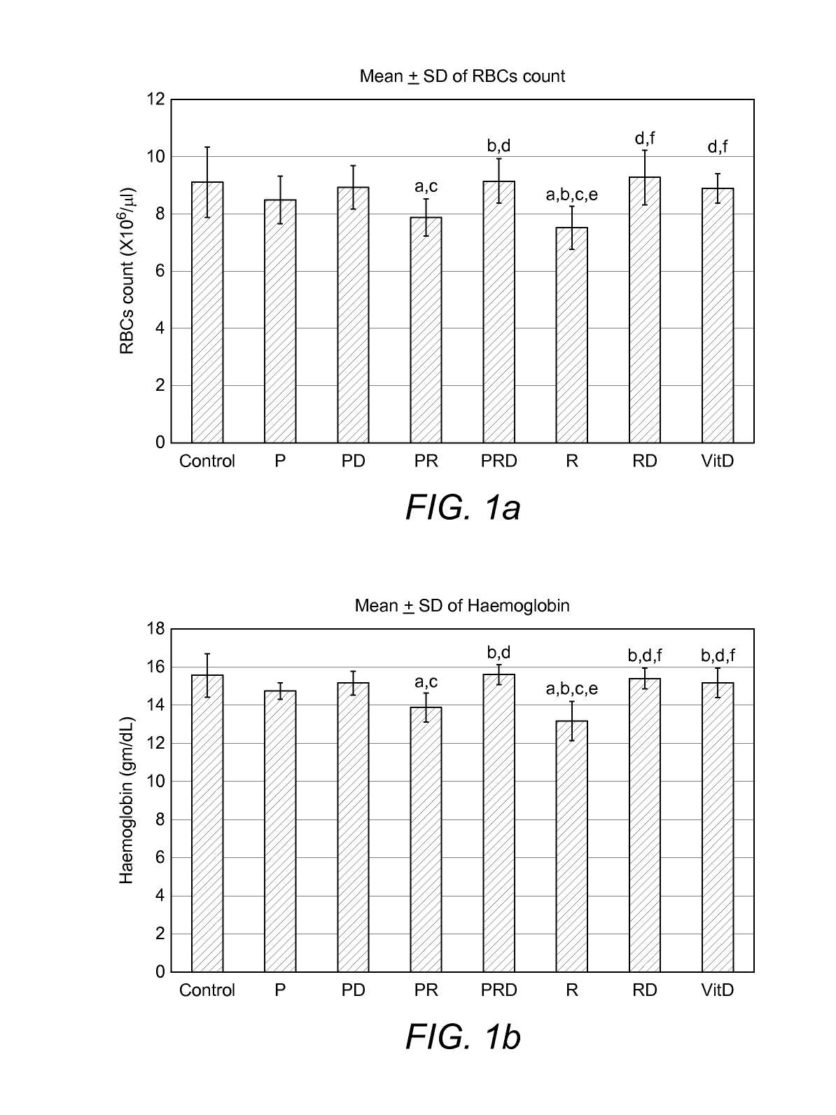

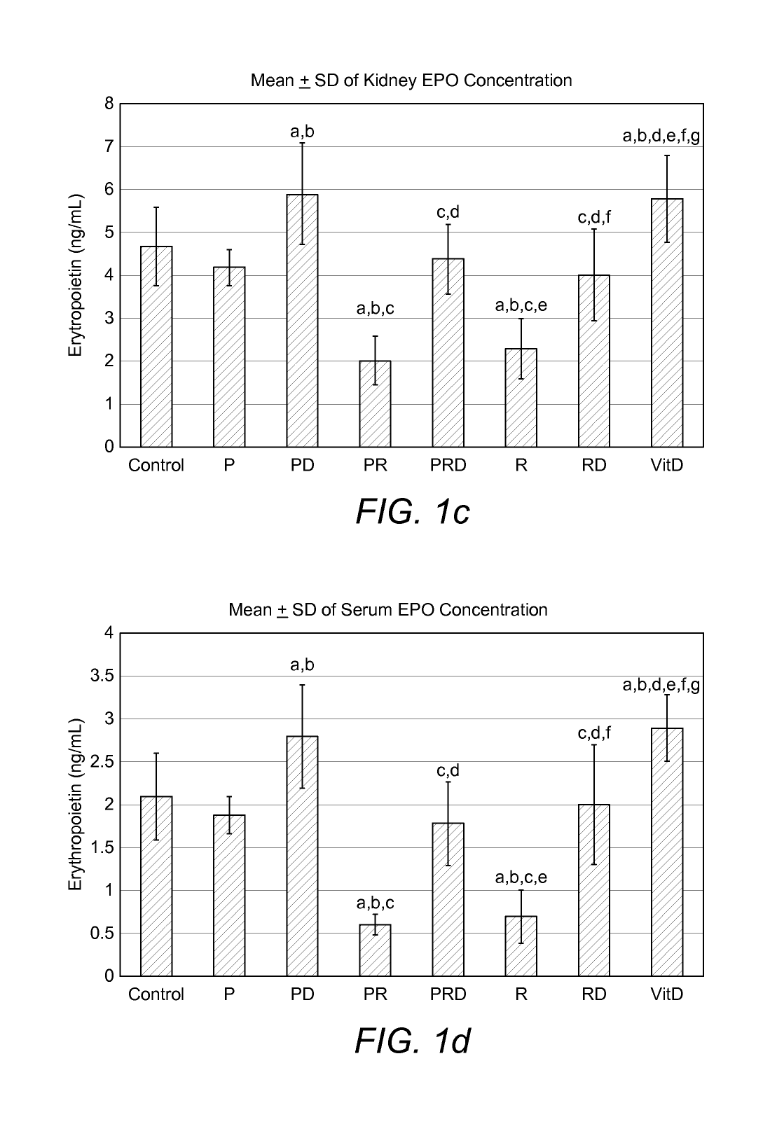

The addition of cholecalciferol prevented the development of anemia in the designated groups as the erythrocyte indices in the rats supplemented with vitamin D3 (PD, PRD, RD and VitD groups) were similar to the control group (p>0.05) and significantly higher (p<0.05) compared to the corresponding non-vitamin D groups (P, PR and R groups) (FIGS. 1(a) & 1(b).

TABLE 1a
Summary of liver enzymes, bilirubin (total and indirect), renal function parameters
and serum 25-OH vitamin D in control, P group, PD group, PR group and PRD group.

| | Control | P Group | PD group | PR group | PRD group |
|---|---|---|---|---|---|
| ALP (IU/L) | 122.6 ± 11.2 | 125.7 ± 9.7 | 120.8 ± 12.4 | 127.3 ± 11.9 | 121.6 ± 11.1 |
| ALT (U/L) | 67 ± 2.4 | 71.2 ± 6.7 | 68.7 ± 4.1 | 66.4 ± 5.3 | 67.3 ± 3.7 |
| AST (U/L) | 77.7 ± 3.9 | 81.8 ± 7.3 | 79.4 ± 5.1 | 75.2 ± 4.9 | 77.9 ± 4.8 |
| Total Bilirubin | 0.5 ± 0.18 | 0.48 ± 0.16 | 0.49 ± 0.11 | 0.51 ± 0.21 | 0.48 ± 0.13 |
| (mg/dL) | | | | | |
| Indirect Bilirubin | 0.15 ± 0.05 | 0.16 ± 0.07 | 0.17 ± 0.05 | 0.19 ± 0.06 | 0.18 ± 0.08 |
| (mg/dL) | | | | | |
| Creatinine (mg/dL) | 0.22 ± 0.06 | 0.2 ± 0.08 | 0.21 ± 0.02 | 0.2 ± 0.03 | 0.21 ± 0.05 |
| Urea (mg/dL) | 47.6 ± 5.1 | 52.3 ± 4 | 47.2 ± 3.8 | 54.6 ± 6.5 | 52.4 ± 7 |
| 25-OH Vitamin D | 43.19 ± 8.1 | 39.6 ± 6.7 | 68.5 ± 9.1^{a,b} | 37.7 ± 9.5^{c} | 65.8 ± 9.1^{a,b,d} |
| (ng/mL) | | | | | |

(^{a}= p < 0.05 compared to control group;
^{b}= p < 0.05 compared to P group;
^{c}= p < 0.05 compared to PD group;
^{d}= p < 0.05 compared to PR group;
e = p < 0.05 compared to PRD group;
f = p < 0.05 compared to R group).

TABLE 1b
Summary of liver enzymes, bilirubin (total and indirect), renal function
parameters and serum 25-OH vitamin D in R group, RD group and VitD group.

| | R group | RD group | Vit D group |
|---|---|---|---|
| ALP (IU/L) | 119 ± 9.9 | 122.8 ± 8.7 | 120.7 ± 12.3 |
| ALT (U/L) | 66.1 ± 2.6 | 69.6 ± 3.3 | 66.7 ± 4.2 |
| AST (U/L) | 78.4 ± 3.2 | 76.8 ± 6.1 | 75.8 ± 4.2 |
| Total Bilirubin | 0.49 ± 0.14 | 0.47 ± 0.2 | 0.51 ± 0.23 |
| (mg/dL) | | | |
| Indirect Bilirubin | 0.17 ± 0.07 | 0.16 ± 0.03 | 0.15 ± 0.08 |
| (mg/dL) | | | |
| Creatinine (mg/dL) | 0.22 ± 0.03 | 0.23 ± 0.07 | 0.2 ± 0.06 |
| Urea (mg/dL) | 47.3 ± 5.8 | 53.9 ± 6.4 | 48.1 ± 5.2 |
| 25-OH Vitamin D | 42 ± 10^{c,e} | 67.9 ± 10.4^{a,b,d,f} | 69.9 ± 9.8^{a,b,d,f} |
| (ng/mL) | | | |

(^{a}= p < 0.05 compared to control group;
^{b}= p < 0.05 compared to P group;
^{c}= p < 0.05 compared to PD group;
^{d}= p < 0.05 compared to PR group;
^{e}= p < 0.05 compared to PRD group;
^{f}= p < 0.05 compared to R group).

Additionally, supplementation with cholecalciferol significantly increased the MCHC in all the treated groups, except for ‘VitD group’, compared to the control and the non-treated groups. However, there was no significant difference in PCV, MCH and MCV between the different study groups (Table 2a and 2b).

### Serum and Kidney Concentrations of EPO

Treatment with RBV, either individually or combined with Peg-INF-α, significantly decreased the levels of EPO at the serum and kidney levels compared to the ‘control’ an P′ groups (Table 2a). On other hand, the addition of cholecalciferol, either alone or in combination with the other drugs, significantly augmented the kidney (p<0.05) and serum (p<0.05) concentrations of the hormone compared to the control and the non-vitamin D (P, PR and R) groups (FIGS. 1(c) and 1(d)).

### Correlations Between Erythrocyte Indices, Serum & Kidney EPO and Serum Vitamin D

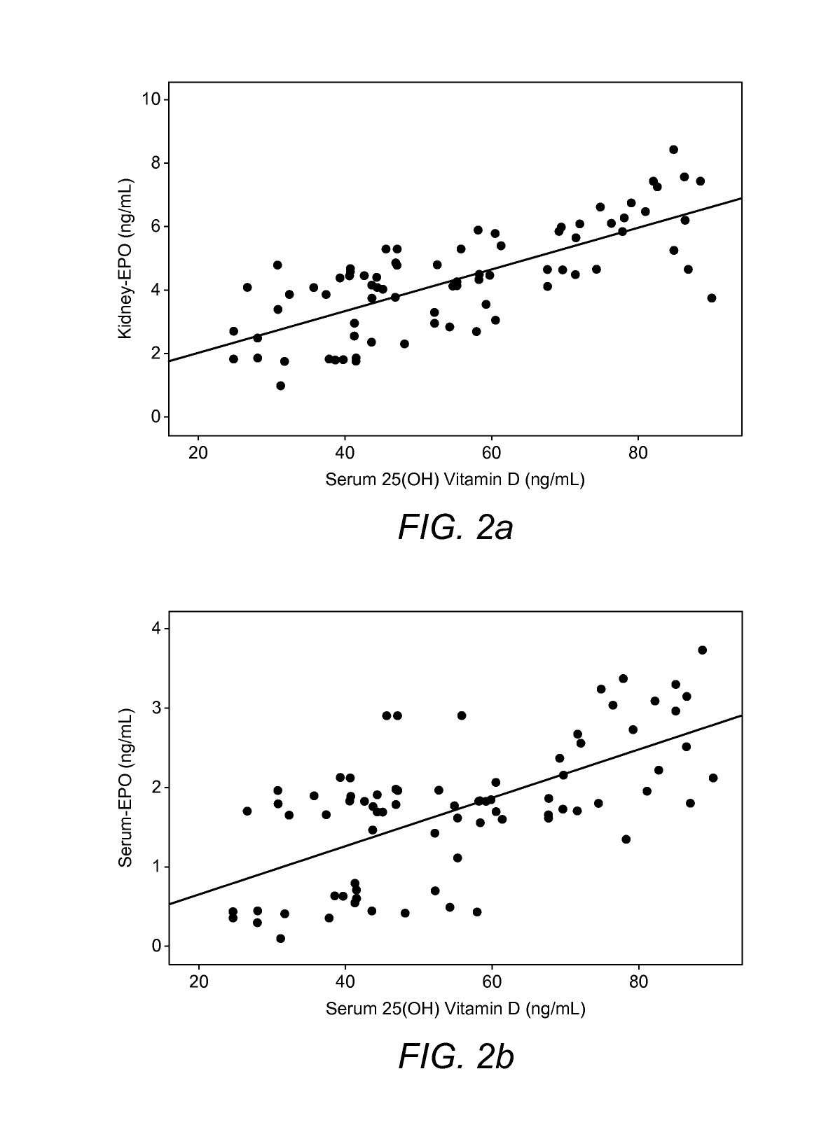

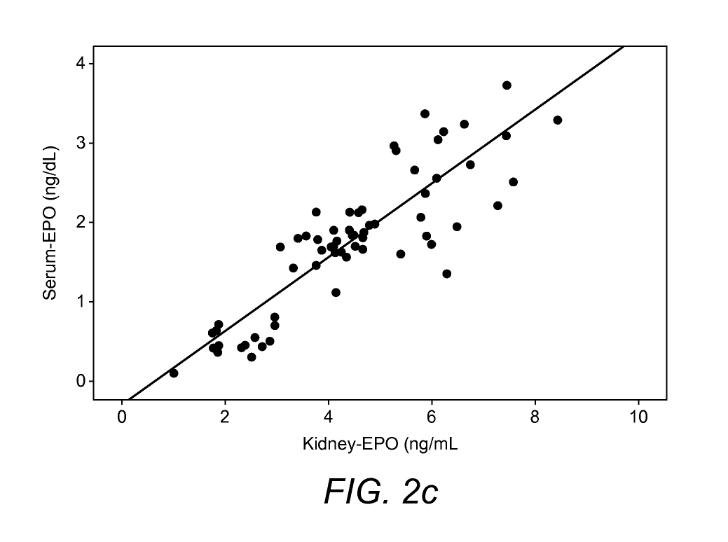

Serum EPO correlated positively and significantly with kidney EPO levels (r=0.874; p=0.2×10⁻⁷). Additionally, there was a significant positive correlation between serum levels of 25-OH vitamin D with serum EPO (r=0.644; p=0.1×10⁻⁶) and kidney EPO concentrations (r=0.736; p=0.2×10⁻⁹) (FIGS. 2(a), 2(b) and 2(c) respectively). Further as shown in FIG. 2(a), R=0.736 and P=0.2×10⁻⁹. As in FIG. 2(b), R=0.644 and P=0.1×10⁻⁶.R=0.874 and P=0.2×10⁻⁷ as shown in FIG. 2(c).

Both renal and serum EPO correlated significantly (p<0.01) with the RBCs count (r=0.33 and 0.335, respectively) and with the hemoglobin concentration (r=0.577 and 0.455, respectively) (Table 3). The RBCs count and hemoglobin also correlated significantly with the serum levels of 25-OH vitamin D (r=0.244 and 0.326, respectively). There was no correlation between serum EPO, kidney EPO and serum 25-OH vitamin D with PCV, MCV and MCH (Table 3).

The current study is the first to report a protective role for vitamin D3 supplementation against the development of RBV-induced anemia by promoting the kidney and serum EPO concentrations in experimental animal model. The study demonstrates a significant decrease in RBCs count and hemoglobin concentration following the use of RBV either individually or in combination with Peg-INF-α. However, there was no significant difference in the values of indirect bilirubin, MCV and MCH between the treated groups and control, suggesting that the treatment lead to the development of normocytic normochromic anemia by suppressing erythropoiesis. This is supported by the observation that RBV±Peg-INF-α significantly decreased the concentrations of EPO at the kidney and serum levels. These observations were detected in the ‘R’ and ‘PR’ groups.

TABLE 2a
Summary of erythrocyte indices, serum and renal concentrations
of EPO in control group, P group, PD group and PR group.

| | Control | P Group | PD group | PR group |
|---|---|---|---|---|
| RBCs (×10⁶/μl) | 9.14 ± 1.2 | 8.5 ± 0.8 | 8.95 ± 0.7 | 7.9 ± 0.6^{a,c} |
| Hb (g/dL) | 15.5 ± 1.07 | 14.7 ± 0.4 | 15.1 ± 0.6 | 13.8 ± 0.7^{a,b,c} |
| PCV (%) | 46.4 ± 7.1 | 42.3 ± 3.1 | 43.1 ± 1.4 | 41.9 ± 1.3 |
| MCV (fL) | 61 ± 3.5 | 62.7 ± 3.3 | 62.1 ± 0.8 | 61.1 ± 2.9 |
| MCH (pg) | 17.6 ± 0.9 | 17.8 ± 0.9 | 17.7 ± 0.4 | 17.5 ± 0.7 |
| MCHC (pg/dL) | 34.7 ± 1 | 33.8 ± 0.5 | 35.9 ± 0.6^{a,b} | 34.5 ± 1^{c} |
| Serum EPO (ng/mL) | 2.1 ± 0.5 | 1.9 ± 0.2 | 2.8 ± 0.6^{a,b} | 0.6 ± 0.13^{a,b,c} |
| Kidney EPO (ng/mL) | 4.7 ± 0.9 | 4.2 ± 0.4 | 5.9 ± 1.2^{a,b} | 2 ± 0.6^{a,b,c} |

(^{a}= p < 0.05 compared to control group;
^{b}= p < 0.05 compared to P group;
^{c}= p < 0.05 compared to PD group;
d = p < 0.05 compared to PR group;
e = p < 0.05 compared to PRD group;
f = p < 0.05 compared to R group and
g = p < 0.05 compared to RD group).

TABLE 2b
Summary of erythrocyte indices, serum and renal concentrations
of EPO in PRD group, R group, RD group and Vit D group.

| | PRD group | R group | RD group | VitD group |
|---|---|---|---|---|
| RBCs (×10⁶/μl) | 9.15 ± 0.8^{b,d} | 7.58 ± 0.7^{a,b,c,e} | 9.3 ± 0.9^{d,f} | 8.9 ± 0.5^{d,f} |
| Hb (g/dL) | 15.54 ± 0.5^{d} | 13.1 ± 1^{a,b,c,e} | 15.3 ± 0.5^{d,f} | 15.1 ± 0.7^{d,f} |
| PCV (%) | 45.1 ± 0.6 | 42.1 ± 2.7 | 42.9 ± 1.3 | 44.9 ± 1.8 |
| MCV (fL) | 59.2 ± 3.6 | 64.3 ± 4.2 | 59.5 ± 1.6 | 61.3 ± 1.8 |
| MCH (pg) | 17.1 ± 0.6 | 17.6 ± 0.7 | 17.4 ± 0.5 | 17.4 ± 0.7 |
| MCHC (pg/dL) | 36.8 ± 1^{a,b,d} | 34.4 ± 0.9^{c,e} | 35.8 ± 0.6^{a,b,d,f} | 34 ± 0.8^{c,e,g} |
| Serum EPO (ng/mL) | 1.8 ± 0.5^{c,d} | 0.7 ± 0.3^{a,b,c,e} | 2 ± 0.7^{c,d,f} | 2.9 ± 0.4^{a,b,d,e,f,g} |
| Kidney EPO (ng/mL) | 4.4 ± 0.8^{c,d} | 2.3 ± 0.7^{a,b,c,e} | 4 ± 1.1^{c,d,f} | 5.8 ± 1^{a,b,d,e,f,g} |

(^{a}= p < 0.05 compared to control group;
^{b}= p < 0.05 compared to P group;
^{c}= p < 0.05 compared to PD group;
^{d}= p < 0.05 compared to PR group;
^{e}= p < 0.05 compared to PRD group;
^{f}= p < 0.05 compared to R group and
^{g}= p < 0.05 compared to RD group).

TABLE 3
Results of correlation analysis using Pearson's
test for serum EPO, kidney EPO an serum 25-OH vitamin
D with RBCs count, haemoglobin concentration, MCV,
MCV, MCH, MCHC and PCV (* = P < 0.05).

| | | RBCs | Hb | MCV | MCH | MCHC | PCV |
|---|---|---|---|---|---|---|---|
| Serum | R | 0.335* | 0.557* | −0.135 | −0.159 | −0.022 | 0.152 |
| EPO | Value | | | | | | |
| | P | 0.002 | 0.1 × 10⁻⁵ | 0.2 | 0.15 | 0.8 | 0.17 |
| | Value | | | | | | |
| Kidney | R | 0.330* | 0.455* | −0.174 | −0.321* | −0.163 | 0.158 |
| EPO | Value | | | | | | |
| | P | 0.003 | 0.1 × 10⁻⁶ | 0.12 | 0.004 | 0.14 | 0.16 |
| | Value | | | | | | |
| 25-OH | R | 0.244* | 0.326* | −0.205 | −0.322* | −0.116 | 0.121 |
| vitamin | Value | | | | | | |
| D | P | 0.02 | 0.003 | 0.069 | 0.004 | 0.3 | 0.2 |
| | Value | | | | | | |

The addition of cholecalciferol (500 IU/day) during the course of the treatment rescued the drugs-induced anemia in all groups (PRD and RD groups) and the erythrocytes parameters were similar to those of the control group. Furthermore, a significant increase in the concentrations of EPO was detected at the serum and kidney levels in all groups treated with vitamin D3. There was also a significant positive correlation between 25-OH vitamin D serum levels with both kidney and serum EPO, RBCs count and hemoglobin concentrations.

Our results suggest that, besides its recently reported potential role in increasing the response rate to Peg-INF-α based therapy, (Bitetto et al, 2010; Abu-Mouch et al, 2011; Nimer and Mouch, 2012) vitamin D could have a potential beneficial role in the prevention of anemia during the treatment of chronic hepatitis C by promoting serum and renal EPO.

The current study correlates with the previous reports as there was no significant change in erythrocyte count and hemoglobin in the ‘P’ group compared to control. Moreover, a significant decrease in the number of RBCs and hemoglobin concentration was observed after 5 weeks of treatment with RBV either individually or in combination with Peg-INF-α. The RBCs count and hemoglobin were significantly lower in the ‘PR’ and ‘R’ groups compared to the ‘Control’ and ‘P’ groups. However, there was no significant changes in indirect bilirubin, MCV and MCH between the RBV±PEG-INF-α treated groups and control, suggesting that the treated rats developed normocytic normochromic anemia. The observed significant decrease in serum and kidney EPO concentrations suggests that the observed anemia with RBV could be due to bone marrow suppression rather than hemolysis.

RBV-induced anemia is primarily dependent on the plasma concentration of the drug rather than the dose/Kg body weight (Poordad et al, 2013; Sulkowski et al, 2013). RBV and its metabolites accumulate in human RBCs, leading to oxidative stress and mitochondrial toxicity and subsequently results in RBCs hemolysis (Sulkowski et al, 2004; Tanaka et al, 2005; Morello et al, 2008; Van Vliebergh et al, 2001; De Franceschi et al, 2000). The uptake rate of RBV by erythrocytes showed dose and species dependency. Canonico et al. (1984a) reported that the largest accumulation of the drug was observed in monkey erythrocyte, followed by human and the lowest accumulation was detected in rat cells. Furthermore, the authors reported that in vitro incubation of erythrocytes from the 3 species with RBV revealed that monkey, human and rat red cells retained 77, 45 and 20% of their initial drug content, respectively (Canonico et al, 1984a). Neither osmotic fragility nor deformability was altered by exposure of red cells to RBV in vitro (Canonico et al, 1984a; 1984b; Cosgriff et al, 1984).

RBV could also induce anemia by inhibiting the process of erythropoiesis through the suppression of bone marrow and decreasing the expression of both EPO and its receptor (Balan et al, 2005; Martin and Jensen, 2008). RBV was also shown to decrease red cell survival as well as inhibit the release of red cell from the bone marrow in monkey and rat (Canonico et al, 1984a; 1984b; Cosgriff et al, 1984; D'Souza and Naryana, 2002; Naryana et al, 2002). However, RBV had no effect on erythrocyte MCV, MCH and MCHC in both species (Canonico et al, 1984a; 1984b; Cosgriff et al, 1984). The administration of Peg-INF-α and RBV in human was also associated with a decrease in serum EPO concentrations (Balan et al, 2005). Therefore, we suggest that RBV, with or without PEG-INF-α, produces normocytic normochromic anemia in rat by suppressing the bone marrow through decreasing the production of EPO from the kidney.

The management of Peg-INF-α and RBV induced anemia during the treatment of CHC was the reduction of RBV dose (Poordad et al, 2013; Sulkowski et al, 2013). However, several published studies have shown that the response rate is associated with higher dose of the drug ((McHutchison et al, 2002; Shiffman et al, 2007a, 2007b; Poordad et al, 2013). As a result, the use of EPO as an erythropoiesis-stimulating agent has been proposed as an alternative for RBV dose reduction to maintain the treatment success rate ((Poordad et al, 2013; Sulkowski et al, 2013).

Vitamin D plays an important role in the process of erythropoiesis and data generated from haemodialysis patients have demonstrated the clinical usefulness of vitamin D supplementation in the treatment of anemia associated with chronic renal failure (Deicher and Horl, 2005; Rianthavorn and Boonyapapong, 2013; Albitar et al, 1997; Saab et al, 2007). It has been demonstrated that vitamin D3 stimulates the proliferation of erythyroid progenitor cells independently from EPO (Deicher and Horl, 2005). It has also been shown that the vitamin D-responsive elements is located on the promoter region of the EPO receptor gene and that vitamin D3 synergize with EPO to increases the production of EPO receptor at the mRNA and protein levels in vitro (Alon et al, 2002). The present study agrees with the previous data as supplementation with cholecalciferol prevented the development of anemia with both Peg-INF-α and RBV and significantly increased endogenous EPO concentrations at the kidney and serum levels.

The aim of the current study was to investigate whether vitamin D has a protective effect against the development of RBV-induced anemia. Nevertheless, measuring the effect of vitamin D supplementation on the cellular expression of EPO protein and EPO receptors, at the protein level using immunohistochemistry or at the gene level using quantitative RT-PCR, in future studies is mandatory to explore the mechanism(s) by which it could promote the action(s) of endogenous EPO during the treatment of CHC.

In conclusion, we suggest that RBV could induce normocytic normochromic anemia by decreasing endogenous EPO at the kidney and serum levels and subsequently suppressing erythropoiesis. Supplementation with vitamin D3 could protect against the associated anaemia with RBV by stimulating the production of endogenous EPO. Further studies are needed to illustrate the clinical value of vitamin D supplementation in the treatment of hepatitis C virus and the prevention of the associated anemia.

Further, in some cases, following the completion of therapy schedule blood samples will be drawn from patients and different serological markers will be evaluated. A serological marker as disclosed may comprise of evaluation of activins and their binding proteins, interferon gamma-inducible protein (IP-10), interleukin (IL-10) among other serological marker. Further, blood concentration of vitamin D and active vitamin D status will also be evaluated following the completion of therapy schedule including supplementation therapy schedule. Genotypic studies as disclosed comprise of studies on genetic variation (if any) in host VDR gene and IL-28 B gene among other related genes. Results will be analyzed following the therapy schedules as disclosed in the present study using known statistical tools. Further, correlation studies will also be conducted in group I, II and III to analyze the correlation (if any) between vitamin D, TQ and routinely used therapy for CHC infection.

The introduction of a complementary treatment protocol with vitamin D and/or TQ in Saudi patients with CHC may improve success rates and achievement of sustained virological response. Additionally, the development of highly sensitive and specific marker(s) such as serological markers, vitamin status markers and genotype markers for treatment outcome may allow early and accurate identification of non-responders before the initiation of the treatment and precise optimization of treatment protocol and duration in CHC.

In another study plan, adult Wistar rats (130 males and 130 females of 280-300 g body weight) may be divided into the following 13 groups (10 males and 10 females per group). Group 1: Normal control, group 2: Peg-INF only, group 3: Peg-INF+Vitamin D3, group 4: Peg-INF+TQ, group 5: Peg-INF+Vitamin D3+TQ, group 6: Peg-INF+Ribavirin, group 7: Peg-INF+Ribavirin+Vitamin D3, group 8: Peg-INF+Ribavirin+TQ, group 9: Peg-INF+Ribavirin+Vitamin D3+TQ, group 10: Ribavirin only, group 11: Ribavirin+Vitamin D3, group 12: Ribavirin+TQ and group 13: Ribavirin+Vitamin D3+TQ. The TQ supplementation therapy comprise of administering the TQ at a dose range of 5 mg/Kg/day to 20 mg/Kg/day, while vitamin D3 supplementation will be at a dose level of 500 IU/day. Vitamin D3 and TQ in the supplementation therapy will be administered orally or other preferred mode of administration. The rats will be injected with the human dose of Peg-INF (3 μg/kg per week) and will be orally administered with the human dose of Ribavirin (12 mg/kg per day) for 3 at least months to induce their adverse effects that are commonly observed when they are used for treatment of human patients with CHC, and also we will measure the effect of vitamin D3±TQ supplementation in preventing the development and/or reducing the severity of these side effects associated with Peg-INF and Ribavirin therapy.

The introduction of a complementary treatment protocol with vitamin D and/or TQ in Saudi patients with CHC may improve success rates and achievement of sustained virological response; and may eliminate/decrease the side effects associated with the currently used treatment of patients with CHC. The present invention will also help in development of highly sensitive and specific marker (s) for treatment outcome of CHC infection and thus may allow early and accurate identification of non-responders and precise optimization of treatment protocol and duration of supplementation therapy in CHC infection.

Although the present embodiments have been described with reference to specific example embodiments, it will be evident that various modifications and changes may be made to these embodiments without departing from the broader spirit and scope of the various embodiments. Accordingly, the specification and drawings are to be regarded in an illustrative rather than a restrictive sense.

### REFERENCES

Averhoff F M, Glass N, and Holtzman D, Global burden of hepatitis C: considerations for healthcare providers in the United States. Clin Infect Dis; 2012; 55 Suppl 1: p. S10-5.
Ghany M G, Strader D B, Thomas D L, and Seeff L B, Diagnosis, management, and treatment of hepatitis C: an update. Hepatology; 2009; 49: p. 1335-74.
McHutchison J G, Manns M P, Brown R S, Jr., Reddy K R, Shiffman M L, and Wong J B, Strategies for managing anemia in hepatitis C patients undergoing antiviral therapy. Am J Gastroenterol; 2007; 102: p. 880-9.
Kowdley K V, Hematologic side effects of interferon and ribavirin therapy. J Clin Gastroenterol; 2005; 39: p. S3-8.
Keeffe E B and Kowdley K V, Hematologic side effects of PEG interferon and ribavirin. Management with growth factors. J Clin Gastroenterol; 2005; 39: p. S1-2.
Kurschel E, Metz-Kurschel U, Niederle N, and Aulbert E, Investigations on the subclinical and clinical nephrotoxicity of interferon alpha-2B in patients with myeloproliferative syndromes. Ren Fail; 1991; 13: p. 87-93.
Sacchi S, Kantarjian H, O'Brien S, Cohen P R, Pierce S, and Talpaz M, Immune-mediated and unusual complications during interferon alfa therapy in chronic myelogenous leukemia. J Clin Oncol; 1995; 13: p. 2401-7.
Tarumi T, Sawada K, Sato N, Kobayashi S, Takano H, Yasukouchi T, et al., Interferon-alpha-induced apoptosis in human erythroid progenitors. Exp Hematol; 1995; 23: p. 1310-8.
Kato K, Kamezaki K, Shimoda K, Numata A, Haro T, Aoki K, et al., Intracellular signal transduction of interferon on the suppression of haematopoietic progenitor cell growth. Br J Haematol; 2003; 123: p. 528-35.
Sulkowski M S, Wasserman R, Brooks L, Ball L, and Gish R, Changes in haemoglobin during interferon alpha-2b plus ribavirin combination therapy for chronic hepatitis C virus infection. J Viral Hepat; 2004; 11: p. 243-50.
Tanaka H, Miyano M, Ueda H, Fukui K, and Ichinose M, Changes in serum and red blood cell membrane lipids in patients treated with interferon ribavirin for chronic hepatitis C. Clin Exp Med; 2005; 5: p. 190-5.
Morello J, Rodriguez-Novoa S, Jimenez-Nacher I, and Soriano V, Usefulness of monitoring ribavirin plasma concentrations to improve treatment response in patients with chronic hepatitis C. J Antimicrob Chemother; 2008; 62: p. 1174-80.
Van Vlierbergh H, Delanghe J R, De Vos M, and Leroux-Roel G, Factors influencing ribavirin-induced hemolysis. J Hepatol; 2001; 34: p. 911-6.
De Franceschi L, Fattovich G, Turrini F, Ayi K, Brugnara C, Manzato F, et al., Hemolytic anemia induced by ribavirin therapy in patients with chronic hepatitis C virus infection: role of membrane oxidative damage. Hepatology; 2000; 31: p. 997-1004.
Balan V, Schwartz D, Wu G Y, Muir A J, Ghalib R, Jackson J, et al., Erythropoietic response to anemia in chronic hepatitis C patients receiving combination pegylated interferon/ribavirin. Am J Gastroenterol; 2005; 100: p. 299-307.
Martin P and Jensen D M, Ribavirin in the treatment of chronic hepatitis C. J Gastroenterol Hepatol; 2008; 23: p. 844-55.
McHutchison J G, Manns M, Patel K, Poynard T, Lindsay K L, Trepo C, et al., Adherence to combination therapy enhances sustained response in genotype-1-infected patients with chronic hepatitis C. Gastroenterology; 2002; 123: p. 1061-9.
Shiffman M L, Salvatore J, Hubbard S, Price A, Sterling R K, Stravitz R T, et al., Treatment of chronic hepatitis C virus genotype 1 with peginterferon, ribavirin, and epoetin alpha. Hepatology; 2007a; 46: p. 371-9.
Shiffman M L, Ghany M G, Morgan T R, Wright E C, Everson G T, Lindsay K L, et al., Impact of reducing peginterferon alfa-2a and ribavirin dose during retreatment in patients with chronic hepatitis C. Gastroenterology; 2007b; 132: p. 103-12.
Poordad F, Lawitz E, Reddy K R, Afdhal N H, Hezode C, Zeuzem S, et al., Effects of ribavirin dose reduction vs erythropoietin for boceprevir-related anemia in patients with chronic hepatitis C virus genotype 1 infection—a randomized trial. Gastroenterology; 2013; 145: p. 1035-1044 e5.
Sulkowski M S, Poordad F, Manns M P, Bronowicki J P, Rajender Reddy K, Harrison S A, et al., Anemia during treatment with peginterferon Alfa-2b/ribavirin and boceprevir: Analysis from the serine protease inhibitor therapy 2 (SPRINT-2) trial. Hepatology; 2013; 57: p. 974-84.
Deicher R and Horl W H, Hormonal adjuvants for the treatment of renal anaemia. Eur J Clin Invest; 2005; 35 Suppl 3: p. 75-84.
Sim J J, Lac P T, Liu I L, Meguerditchian S O, Kumar V A, Kujubu D A, et al., Vitamin D deficiency and anemia: a cross-sectional study. Ann Hematol; 2010; 89: p. 447-52.
Rianthavorn P and Boonyapapong P, Ergocalciferol decreases erythropoietin resistance in children with chronic kidney disease stage 5. Pediatr Nephrol; 2013; 28: p. 1261-6.
Alon D B, Chaimovitz C, Dvilansky A, Lugassy G, Douvdevani A, Shany S, et al., Novel role of 1,25(O H)(2)D(3) in induction of erythroid progenitor cell proliferation. Exp Hematol; 2002; 30: p. 403-9.
Albitar S, Genin R, Fen-Chong M, Serveaux M O, Schohn D, and Chuet C, High-dose alfacalcidol improves anaemia in patients on haemodialysis. Nephrol Dial Transplant; 1997; 12: p. 514-8.
Saab G, Young D O, Gincherman Y, Giles K, Norwood K, and Coyne D W, Prevalence of vitamin D deficiency and the safety and effectiveness of monthly ergocalciferol in hemodialysis patients. Nephron Clin Pract; 2007; 105: p. c132-8.
S alum E, Kampus P, Zilmer M, Eha J, Butlin M, Avolio A P, et al., Effect of vitamin D on aortic remodeling in streptozotocin-induced diabetes. Cardiovasc Diabetol; 2012; 11: p. 58.
Bitetto D, Fabris C, Falleti E, Fornasiere E, Fumolo E, Fontanini E, et al., Vitamin D and the risk of acute allograft rejection following human liver transplantation. Liver Int; 2010; 30: p. 417-44.
Abu-Mouch S, Fireman Z, Jarchovsky J, Zeina A R, and Assy N, Vitamin D supplementation improves sustained virologic response in chronic hepatitis C (genotype 1)-naive patients. World J Gastroenterol; 2011; 17: p. 5184-90.
Nimer A and Mouch A, Vitamin D improves viral response in hepatitis C genotype 2-3 naive patients. World J Gastroenterol; 2012; 18: p. 800-5.
Maddrey W C, Safety of combination interferon alfa-2b/ribavirin therapy in chronic hepatitis C-relapsed and treatment-naive patients. Semin Liver Dis; 1999; 19 Suppl 1: p. 67-75.
Jacobson I M, Gonzalez S A, Ahmed F, Lebovics E, Min A D, Bodenheimer H C, Jr., et al., A randomized trial of pegylated interferon alpha-2b plus ribavirin in the retreatment of chronic hepatitis C. Am J Gastroenterol; 2005; 100: p. 2453-62.
Fried M W, Shiffman M L, Reddy K R, Smith C, Marinos G, Goncales F L, Jr., et al., Peginterferon alfa-2a plus ribavirin for chronic hepatitis C virus infection. N Engl J Med; 2002; 347: p. 975-82.
Canonico P G, Kastello M D, Spears C T, Brown J R, Jackson E A, and Jenkins D E, Effects of ribavirin on red blood cells. Toxicol Appl Pharmacol; 1984a; 74: p. 155-62.
Canonico P G, Kastello M D, Cosgriff T M, Donovan J C, Ross P E, Spears C T, et al., Hematological and bone marrow effects of ribavirin in rhesus monkeys. Toxicol Appl Pharmacol; 1984b; 74: p. 163-72.
Cosgriff T M, Hodgson L A, Canonico P G, White J D, Kastello M D, Donovan J C, et al., Morphological alterations in blood and bone marrow of ribavirin-treated monkeys. Acta Haematol; 1984; 72: p. 195-200.
D'Souza U J and Narayana K, Mechanism of cytotoxicity of ribavirin in the rat bone marrow and testis. Indian J Physiol Pharmacol; 2002; 46: p. 468-74.
Narayana K, D'Souza U J, and Seetharama Rao K P, The genotoxic and cytotoxic effects of ribavirin in rat bone marrow. Mutat Res; 2002; 521: p. 179-85.

What is claimed is:

1. A method, comprising:
diagnosing patients with a chronic hepatitis C (CHC) infection;
administering a regularly used an anti-CHC infection drug;
administering a supplemental therapy wherein the supplementation therapy comprise of a vitamin D; and
analyzing a CHC infection marker to monitor the treatment effect of supplemental therapy.

2. The method of claim 1, wherein the supplemental therapy comprise of administration of vitamin D3.

3. The method of claim 1, wherein the anti-CHC infection drug comprise of administering a pegylated interferon-α (Peg-INFα).

4. The method of claim 1, wherein the anti-CHC infection drug comprise of administering a ribavirin (RBV).

5. The method of claim 3, wherein the Peg-INFα is Peg-INFα-2a.

6. The method of claim 1, wherein monitoring will comprise of comparing the result of the patient receiving supplemental therapy with the result from the patient not receiving the supplemental therapy.

7. The method of claim 1, wherein the anti-CHC infection drug comprise of administering a Peg-INFα along with the RBV.

8. A method, comprising:
diagnosing patients with a chronic hepatitis C (CHC) infection;
administering a regularly used an anti-CHC infection drug;
administering a supplemental therapy wherein the supplemental therapy comprise of a vitamin D; and
analyzing a development of anemia following the supplemental therapy administration as compared to the patient not receiving the supplemental therapy.

9. The method of group 8, wherein a hemotological profile will be examined following the supplemental therapy.

10. The method of claim 8, wherein the supplemental therapy comprise of administration of vitamin D3.

11. The method of claim 8, wherein the anti-CHC infection drug comprise of administering a pegylated interferon-α (Peg-INFα).

12. The method of claim 8, wherein the anti-CHC infection drug comprise of administering a ribavirin (RBV).

13. The method of claim 11, wherein the Peg-INFα is Peg-INFα-2a.
